(19) United States
(12) Patent Application Publication
(10) Pub. No.: US 2011/0270078 A1
(43) Pub. Date: Nov. 3, 2011

(54) METHODS AND SYSTEMS OF COMBINING MAGNETIC RESONANCE AND NUCLEAR IMAGING

(76) Inventors: Douglas J. Wagenaar, Westlake Village, CA (US); Bradley E. Patt, Sherman Oaks, CA (US); Dirk Meier, Bekkestua (NO); James W. Hugg, Simi Valley, CA (US)

(21) Appl. No.: 13/099,202

(22) Filed: May 2, 2011

Related U.S. Application Data
(60) Provisional application No. 61/330,310, filed on Apr. 30, 2010.

(51) Int. Cl. A61B 5/055

(52) U.S. Cl. 600/411

(57) ABSTRACT

An multi-modality imaging system for imaging of an object under study that includes a magnetic resonance imaging (MRI) apparatus and an MRI-compatible single-photon nuclear imaging apparatus imbedded within the RF coil of the MRI system such that sequential or simultaneous imaging can be done with the two modalities using the same support bed of the object under study during the imaging session.

### CROSS-REFERENCE TO RELATED APPLICATIONS

The present application claims benefit under 37 C.F.R §1.78 of U.S. Provisional Application No. 61/330,310, filed Apr. 30, 2010, and entitled “Magnetic Resonance RF Coil with Nuclear Imaging Capability,” the entire contents of which are incorporated by reference herein. The present application also incorporates the entire contents of U.S. Pat. No. 7,629,586, issued Dec. 8, 2009, and U.S. Patent Application No. 2010/0072377, both entitled “Methods and systems of combining magnetic resonance and nuclear imaging,” by reference herein.

### STATEMENT REGARDING FEDERALLY SPONSORED RESEARCH OR DEVELOPMENT

This invention was made at least in part with U.S. Government support under National Institutes of Health (NIH) Small Business Innovation Research (SBIR) Grant No. R44-EB006712. The U.S. Government may have certain rights to this invention.

### FIELD OF THE INVENTION

The invention relates generally to multi-modality medical imaging. More particularly, the invention relates to methods and systems for combining magnetic resonance imaging (MRI) with single photon emission (SPE) imaging, such as single photon emission computed tomography (SPECT).

### BACKGROUND OF THE INVENTION

Magnetic resonance imaging is a technique used to visualize the inner volume of an object (e.g., a human or animal body or a body part or tissue specimen or a test phantom). Magnetic field strengths for MRI studies of humans typically require 1.5 or 3.0 Tesla (T) and studies of animals typically require 4.7 or 7.0 T, although magnets up to 17 T have been reported. Organ-specific radio frequency (RF) coils are routinely used in neurology, mammography, cardiology, and urology applications. Additionally, RF coils for specific orthopaedic imaging applications such as shoulder or knee evaluation are used in clinical radiology. In more general applications, a whole-body or head volume RF coil may be used.

Single photon emission computed tomography (SPECT) is a nuclear medicine tomographic imaging technique using gamma rays. Conventionally, this imaging technique accumulates counts of gamma photons that are absorbed by a scintillator crystal. The crystal scintillates in response to photoelectric or Compton scattering interactions with gamma photons to produce prompt fluorescent emission of light photons. Photomultiplier tubes (PMTs) behind the scintillator crystal detect the fluorescent photons, and a computer sums the fluorescent counts. The computer in turn constructs and displays an image of the relative spatial count density on a monitor. This planar image then reflects the distribution and relative concentration of radioactive tracer elements present in the organs and tissues imaged as seen from one unique perspective. Tomographic, 3-dimensional information about the distribution and concentration of radioactive tracer is obtained by changing the unique perspective of the planar nuclear imager in small angular increments, conventionally by having the imaging detector follow a circular orbital path around the patient.

Although there may be clinical benefits to combine SPECT and MRI images, any prospect of combining SPECT and MRI within a single system has been mostly dismissed because the functions of the PMTs in a typical SPECT system are severely compromised by the high magnetic fields needed for MRI and because magnetic field uniformity needed for MRI is distorted by the electrically conducting components in the typical SPECT system. The PMTs are typically packed edge-to-edge on a plane to form a rectangular-shaped Anger-type camera that typically occupies a large volume of about 40 cm (width)×60 cm (length)×30 cm (height). This large volume occupied by the typical Anger camera used in SPECT is another impediment to its use within the bore of an MRI imaging system.

Recent advances in semiconductor technology have opened the possibility of replacing the PMTs and the scintillator crystal of a SPECT system with a semiconductor detector, such as cadmium zinc telluride (CdZnTe or CZT) detector. The CZT detector operates in the magnetic field inside a MR imaging apparatus. The CZT detector is referred to as a direct detector of radiation and operates by producing negative and positive charges (electrons and holes) through interaction with gamma photons. However, using a CZT detector for detecting gamma photons in a strong magnetic field is still not a trivial task because the electrons and holes of the CZT detector need to travel distances of 5 mm or more within the CZT crystal to generate signals on the anode and/or cathode surfaces of the CZT detector. In most geometric orientations of the CZT detector relative to the direction of the magnetic field, the charge-carrier drift is subject to the Lorentz force which may distort the generated SPECT imaging signals.

In addition, it may be considered necessary to remove the electronics for signal amplification, address generation, logical operations, and other processing functions from the CZT module (in the high magnetic field) and to bring these electronics to a more distant location (in which a lower field can be found), thereby removing a cause of interference (e.g., either the offending electronics does not function in the high field or the offending electronics causes the MRI to have artifacts). However, locating the electronics away from the magnetic field requires that they be connected via relatively long cables that result in an increased noise and signal distortion. Furthermore, these cables need to be shielded and low-pass filtered to prevent introduction of RF noise into the MRI system.

In view of the foregoing and as discussed in Wagenaar, et al. “Rationale for the Combination of Nuclear Medicine with Magnetic Resonance for Pre-clinical Imaging,” Technology in Cancer Research and Treatment, 2006, Vol. 5, pp. 343-350, which is incorporated by reference herein in its entirety, it would be desirable to combine MRI with SPE imaging, such as SPECT, to provide a more informative image consisting of both high resolution, anatomical imaging (provided by MRI) and molecular imaging (provided by SPECT). Of course, such a SPECT system must occupy a small volume so that it can fit into the bore of the MRI system.

The above information disclosed in this Background section is only for enhancement of understanding of the background of the invention and therefore it may contain information that does not form the prior art that is already known to a person of ordinary skill in the art.

### SUMMARY OF THE INVENTION

An aspect of the present invention provides a dual-modality, co-registered, and optionally fused image dataset from MRI and single-photon emission (SPE) nuclear medicine imaging modalities in a single imaging session. The single dual-modality imaging session allows a body (e.g., a human or animal body), body part (e.g., head, knee, breast), organ (e.g., heart, prostate, thyroid) or other object being scanned to remain essentially motionless relative to the two imaging systems for sequential scanning while using the same body position on the same bed, thereby reducing or minimizing mis-registration artifacts from changes in body orientation between the imaging studies. In this case, the SPE system could be located adjacent to the MRI system and in its fringe magnetic field and the two fields of view would not coincide. It is also possible for the SPE system to be located inside the MRI system, but not at the center of the MRI field of view.

Alternatively, the single dual-modality imaging session can include the concurrent or simultaneous operation of the two imaging modalities, providing exact co-registration in spatial position as well as in time. In this alternate case, the SPE system would be located inside the MRI system and the field of view of both imaging systems would coincide. The ability to perform co-registered and optionally fused dual-modality imaging may be helpful in either clinical or pre-clinical studies for the development of drugs or therapies or the general study of biological processes.

According to an embodiment of the present invention, a combined MRI and SPE imaging system includes MRI-compatible gamma photon detectors, collimators, and an MRI system with an RF coil. The combination of gamma photon detectors and collimators is required for SPE imaging. The coupled collimators and detectors detect some of the gamma photons emitted by the object under study and generate a direct detection signal that is transmitted to electronics designed to process the detection signal, typically determining the time, energy, and position of each gamma photon event. The RF coil is required for MRI. Here, the MRI-compatible gamma photon detector is configured to make an SPE image of the object under study under an influence of the magnetic field suitable for MRI. The RF coil and gamma photon detector and/or collimator are mechanically integrated in this embodiment of invention.

In one embodiment of the system, each MRI-compatible gamma photon detector is made of a semiconductor substrate material, such as silicon (Si), germanium (Ge), cadmium telluride (CdTe), mercuric iodide (HgI₂), thallium bromide (TlBr), gallium arsenide (GaAs), cadmium zinc telluride (CdZnTe or CZT), or cadmium manganese telluride (CdMnTe). Such direct-conversion detectors have a plurality of electrodes to collect the charge carriers (electrons and holes) generated by gamma photons incident upon and penetrating into the detector.

In another embodiment of the system, each MRI-compatible gamma photon detector includes a scintillator substrate and an MRI-compatible optical photon detector such as a photo-diode (PD), avalanche photo-diode (APD), solid-state photomultiplier (SSPM or SiPM), and multi-channel plate (MCP). The scintillator and photon detector may be coupled by a light guide.

In one embodiment of the system, each MRI-compatible gamma photon detector and collimator combination together forms a collimator-detector unit (CDU). Each CDU acquires a projection image. When used together with other similar or identical CDUs that can be arranged at different angular orientations relative to the first CDU, the total of all CDU projection images form the basis for tomographic 3-dimensional image reconstruction.

In one embodiment of the system, the MRI-compatible gamma photon detectors remain stationary during the SPE image acquisition; alternatively, the gamma photon detectors and/or collimators and/or RF coil can rotate or otherwise move to various positions to provide additional project views during the SPE image acquisition.

In one embodiment of the system, the imaging system includes an RF coil with a plurality of CDUs imbedded within the RF coil such that the RF coil/CDU combination forms a single mechanically integrated imaging accessory that can be used for dual-modality imaging of the object for which the RF coil alone was designed. The MRI and SPE imaging can be either sequential (with bed motion to move the object) or substantially simultaneous.

In one embodiment of the system, the imaging system further includes a correction processor adapted to compensate for a Lorentz-force effect on the charge carriers traveling within the at least one semiconductor substrate and under the influence of the magnetic field suitable for MRI.

In another embodiment of the present invention, a method of combining MRI and SPE imaging is provided. The method includes: introducing (e.g., injection or ingestion) a radioactive isotope into an object under study; detecting gamma photons from the radioactive isotope within the object under study by an MRI-compatible detector; SPE imaging the object under study with a plurality of CDUs imbedded within the RF coil of the MRI system; and simultaneously or sequentially magnetic resonance (MR) imaging the object under study with at least one RF coil positioned around or adjacent to the object. Here, the object under study is SPE imaged under an influence of the magnetic field suitable for MRI.

According to another embodiment of the present invention, a combined magnetic resonance imaging (MRI) and single-photon emission (SPE) imaging system is provided. The imaging system includes: an MRI system including at least one SPE-compatible radiofrequency (RF) coil, the MRI system being for magnetic resonance (MR) imaging of an object; and an SPE imaging system including at least one MRI-compatible gamma photon detector and at least one MRI-compatible collimator, the SPE imaging system being for SPE imaging of the object. Here, the at least one SPE-compatible RF coil is mechanically integrated with the at least one MRI-compatible gamma photon detector and/or the at least one MRI-compatible collimator.

In one embodiment, the MRI system and the SPE imaging system of the combined MRI and SPE imaging system are configured to produce sequential and/or simultaneous images of the object.

In one embodiment, the SPE imaging system is configured to operate inside an imaging magnetic field of the MRI system.

In one embodiment, the SPE imaging system is configured to operate outside the MRI system and positioned in a fringe magnetic field of the MRI system.

In one embodiment, the SPE imaging system is configured to produce at least one projection image and/or at least one SPE computed tomographic (SPECT) image.

In one embodiment, the MRI system includes a compensator configured to compensate for the presence of the SPE imaging system, the compensator including an electromagnetic shield, a resonant element tuner, a static and/or dynamic magnetic field shimmer, an eddy-current compensator, an electromagnetic load compensator, a cooler, a power transmission filter, and/or a data transmission filter.

In one embodiment, the at least one SPE-compatible RF coil is selected from the group consisting of surface coil, volume coil, multi-channel array coil, parallel transmit coil, and parallel receive coil.

In one embodiment, the SPE imaging system includes a compensator configured to compensate for the presence of the MRI system, the compensator including an electromagnetic shield, a Lorentz effect compensator, an electromagnetic load compensator, a cooler, a power transmission filter, and/or a data transmission filter.

In one embodiment, the at least one MRI-compatible gamma photon detector includes a direct-conversion substrate material selected from the group consisting of silicon (Si), germanium (Ge), cadmium telluride (CdTe), mercuric iodide (HgI₂), thallium bromide (TlBr), gallium arsenide (GaAs), cadmium zinc telluride (CdZnTe or CZT), and cadmium manganese telluride (CdMnTe). Here, the at least one MRI-compatible gamma photon detector may include: at least one direct-conversion substrate for producing charge carriers through interaction with gamma photons; and a plurality of electrodes for collecting the charge carriers.

In one embodiment, the at least one MRI-compatible gamma photon detector includes: at least one scintillator substrate for producing optical photons through interaction with gamma photons; and at least one MRI-compatible optical photon detector for producing an electrical signal. Here, the at least one MRI-compatible optical photon detector may include photodiodes, solid-state photomultipliers, and/or multi-channel plates.

In one embodiment, the MRI system is configured to provide information to the SPE system to improve a SPE computed tomographic (SPECT) image reconstruction, the SPE system including an attenuation compensator, a scattering compensator, and/or a statistical reconstructor.

In one embodiment, the at least one MRI-compatible collimator is configured to have a single pinhole, multiple pinholes, parallel multiple holes, converging multiple holes, or diverging multiple holes; or is configured to be an inverse collimator composed of parallel, converging, or diverging multiple pins; or is configured to have multiple hole coded apertures, slits and/or slats; or is configured to have rotating slits and/or slats; or is configured to be an electronic (Compton camera) collimator.

In one embodiment, the at least one MRI-compatible collimator includes a substrate of gamma photon attenuating material with electromagnetic conductivity and susceptibility properties that do not distort main and RF magnetic fields beyond the capability of the MRI system to compensate.

In one embodiment, the at least one MRI-compatible gamma photon detector and/or the at least one MRI-compatible collimator are at least partially embedded into the contiguous volume enclosing the at least one SPE-compatible RF coil.

In one embodiment, the at least one SPE-compatible RF coil is at least partially embedded into the contiguous volume enclosing the at least one MRI-compatible gamma photon detector and/or the at least one MRI-compatible collimator.

In one embodiment, the at least one SPE-compatible RF coil is supported on the at least one MRI-compatible gamma photon detector and/or the at least one MRI-compatible collimator.

In one embodiment, the at least one MRI-compatible gamma photon detector and/or the at least one MRI-compatible collimator are supported on the at least one SPE-compatible RF coil.

In one embodiment, the SPE imaging system is configured to be stationary during imaging.

In one embodiment, the SPE imaging system is configured to provide motion to the at least one MRI-compatible gamma photon detector and/or the at least one MRI-compatible collimator and/or the at least one SPE-compatible RF coil.

According to another embodiment of the present invention, a method of combining magnetic resonance imaging (MRI) and single-photon emission (SPE) imaging is provided. The method includes: introducing a radioactive isotope into an object; acquiring at least one MR image or spectrum of an object utilizing an MRI system including at least one SPE-compatible radiofrequency (RF) coil; and acquiring at least one SPE image of the object utilizing an SPE imaging system including at least one MRI-compatible gamma photon detector and at least one MRI-compatible collimator; wherein the at least one SPE-compatible RF coil is mechanically integrated with the at least one MRI-compatible gamma photon detector and/or the at least one MRI-compatible collimator.

In one embodiment, the MRI system and the SPE imaging system of the combined MRI and SPE imaging system produce sequential and/or simultaneous images of the object.

In one embodiment, the SPE imaging system is stationary during imaging.

In one embodiment, the SPE imaging system provides for motion of the at least one MRI-compatible gamma photon detector and/or the at least one MRI-compatible collimator and/or the at least one SPE-compatible RF coil.

According to another embodiment of the present invention, a device for combined magnetic resonance imaging (MRI) and single-photon emission (SPE) imaging is provided. The device includes at least one SPE-compatible radiofrequency (RF) coil mechanically integrated with at least one MRI-compatible gamma photon detector and/or at least one MRI-compatible collimator.

In one embodiment, the at least one SPE-compatible RF coil is selected from the group consisting of surface coil, volume coil, multi-channel array coil, parallel transmit coil, and parallel receive coil.

In one embodiment, the at least one MRI-compatible gamma photon detector includes a direct-conversion substrate material selected from the group consisting of silicon (Si), germanium

(Ge), cadmium telluride (CdTe), mercuric iodide (HgI₂), thallium bromide (TlBr), gallium arsenide (GaAs), cadmium zinc telluride (CdZnTe or CZT), and cadmium manganese telluride (CdMnTe).

In one embodiment, the at least one MRI-compatible gamma photon detector includes: at least one direct-conversion substrate for producing charge carriers through interaction with gamma photons; and a plurality of electrodes for collecting the charge carriers.

In one embodiment, the at least one MRI-compatible gamma photon detector includes: at least one scintillator substrate for producing optical photons through interaction with gamma photons; and at least one MRI-compatible optical photon detector for producing an electrical signal. The at least one MRI-compatible optical photon detector may include photodiodes, solid-state photomultipliers, and/or multi-channel plates.

In one embodiment, the at least one MRI-compatible collimator is configured to have a single pinhole, multiple pinholes, parallel multiple holes, converging multiple holes, or diverging multiple holes; or is configured to be an inverse collimator composed of parallel, converging, or diverging multiple pins with no holes; or is configured to have multiple hole coded apertures, slits and/or slats; or is configured to have rotating slits and/or slats; or is configured to be an electronic (Compton camera) collimator.

In one embodiment, the at least one MRI-compatible collimator includes a substrate of gamma photon attenuating material with electromagnetic properties, such as conductivity and susceptibility, that do not distort main and RF magnetic fields beyond the capability of the MRI system to compensate.

In one embodiment, the at least one MRI-compatible gamma photon detector and/or the at least one MRI-compatible collimator are at least partially embedded into the contiguous volume enclosing the at least one SPE-compatible RF coil.

In one embodiment, the at least one SPE-compatible RF coil is at least partially embedded into the contiguous volume enclosing the at least one MRI-compatible gamma photon detector and/or the at least one MRI-compatible collimator.

In one embodiment, the at least one SPE-compatible RF coil is supported on the at least one MRI-compatible gamma photon detector and/or the at least one MRI-compatible collimator.

In one embodiment, the at least one MRI-compatible gamma photon detector and/or the at least one MRI-compatible collimator are supported on the at least one SPE-compatible RF coil.

These and other features and aspects of the present invention will be more fully understood when considered with respect to the following detailed description, appended claims, Wagenaar et al., US Patent Application No. 2010/0072377, Wagenaar et al., U.S. Pat. No. 7,629,586, and accompanying drawings.

### BRIEF DESCRIPTION OF THE DRAWINGS

The accompanying drawings, together with the specification, illustrate exemplary embodiments of the present invention, and, together with the description, serve to explain the principles of the present invention.

FIG. 1 illustrates the configuration of a SPE collimator-detector unit or CDU, in accordance with an embodiment of the present technique.

FIG. 2 illustrates the use of a SPECT collimator-detector unit, CDU in use to create a projection image, in accordance with an embodiment of the present technique.

FIG. 3 illustrates a generalized surface RF coil that is used in an MRI system, in accordance with an embodiment of the present technique.

FIG. 4 illustrates a generalized surface RF coil with four CDUs imbedded at various angles relative to each other such that 4 different projection views are obtained to form a tomographic image dataset, in accordance with an embodiment of the present technique.

FIG. 5 illustrates embedding of individual CDUs with a cardiac RF coil, in accordance with an embodiment of the present technique.

### DETAILED DESCRIPTION

In the following detailed description, only certain exemplary embodiments of the present invention are shown and described, by way of illustration. As those skilled in the art will recognize, the described exemplary embodiments may be modified in various ways, all without departing from the spirit or scope of the present invention. Accordingly, the drawings and description are to be regarded as illustrative in nature, and not restrictive.

An embodiment of the present invention is designed to enhance or augment MRI imaging by incorporating an additional modality within an RF coil for sequential or simultaneous operation of the modality with an MRI system. The added modality is tomographic SPECT or planar imaging based on the single-photon emission (SPE) radiotracer principle.

In one embodiment of the present invention, the SPE imaging system is based on a semiconductor direct conversion detector, such as a cadmium zinc telluride (CZT) detector. The embodiment of the present invention reduces the possibility of organ or body part misalignment between MRI and SPE scans by incorporating the two modalities into one imaging session. The embodiment avoids the changing of the position of the human or animal or object being imaged, and ensures the accuracy of the co-registration between the data acquired from the two modalities. It allows for the concurrent or simultaneous acquisition of dynamic and/or static data sets and the single-injection of combined contrast agents for the two modalities. Moreover the SPE imaging system data are not detrimentally affected by the magnetic fields produced by the MRI scanner (or imaging system) and vice versa.

In more detail, conventional nuclear medicine imaging relies on the use of PMTs to detect fluorescent light emission from the absorption of gamma photons in scintillator crystals. As discussed above, the PMTs, however, do not work in magnetic fields. In one embodiment of the present invention, by replacing the scintillator and PMT combination with a solid-state semiconductor detector, such as a CdZnTe or CZT detector, the embodiment of the present invention realizes a gamma camera that can operate in the magnetic field inside a MRI apparatus. Alternatively, another embodiment of the invention provides for use of a scintillating crystal with replacement of the PMT by an MRI-compatible photo-detector, such as an avalanche photo-diode (APD) or solid-state silicon photomultiplier (SiPM or SSPM).

Multimodality imaging offers many opportunities for the combination of spatially and temporally-registered data. One embodiment of the present invention combines anatomical context and functional information, such as the anatomical delineation of the boundaries of a tumor (using, e.g., MRI) with the functional definition of aggressive cancer cells at the perimeter and necrotic cells at the core of the tumor (using, e.g., SPECT). This is but one of many possible combinations of imaging data, and the present invention is not thereby limited. In one embodiment of the present invention, the combination of MRI data with single-photon nuclear imaging data with spatial and temporal registration is realized through the use of the semiconductor nature of the CZT in order to overcome the magnetic field limitations of conventional PMTs.

Co-registered images may lose some of their precision if organs or body parts are located at different positions (i.e., they have shifted) during the imaging sessions. As such, one embodiment of the present invention includes a semiconductor CZT detector that can sequentially or simultaneously provide SPE imaging (e.g., SPECT) and MRI imaging because the semiconductor CZT imaging detector can operate in a magnetic field, whereas the PMT-based imaging devices cannot operate in a magnetic field. That is, simultaneous imaging is possible because the SPE imaging (or SPECT) system of an embodiment of the present invention is located inside the field of the MRI system and the SPE detectors are imbedded within the RF coil of the MRI system.

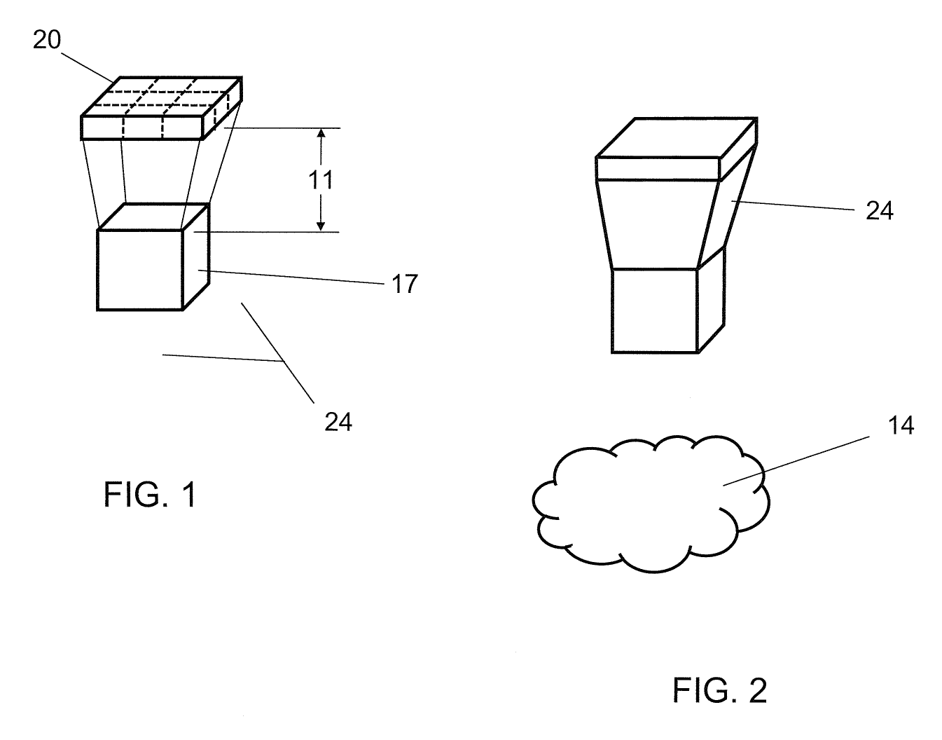

FIG. 1 illustrates the configuration of a SPE collimator-detector unit or CDU, composed of one or more semiconductor imaging detectors 20 arranged in a plane as shown or, if more than one semiconductor imaging detector is used, non-planar arrangements are also envisioned. A high-atomic number material such as lead, tungsten, bismuth, gold, tantalum, or platinum (but not excluding other heavy metals) can be used to form the collimator 17. The collimator can be formed as parallel holes in a honeycomb (hexagonal) pattern or in a square array pattern. The holes can be circular, square, or hexagonal-shaped. The holes can be matched one hole-for-one pixel of the semiconductor detector. The collimator holes can converge to a focal point or diverge to create a larger field of view than the CZT area. The collimator can have a single pinhole or multiple pinholes with overlapping or non-overlapping projections onto the semiconductor detector. The collimator can have large numbers (>20) of holes arranged in a pseudo-random fashion to form a coded aperture pattern.

The collimator can also be active, as in a Compton camera, where electronic “collimation” is provided by replacing the collimator 17 by a second detector (such as silicon or germanium) that is efficient for Compton scattering. The gamma photons first scatter in the “collimating” detector, which is pixellated to determine the position of the scatter event, and then are absorbed in the main detector, where both the energy and position are again determined. The distance 11 between the collimator exit surface and the detector entrance surface for photons emitted from the object being imaged can be negligible or several centimeters, depending upon whether a multiple hole collimator or a pinhole collimator are in use.

Alternatively, the detector 20 may comprise a scintillating crystal (either monolithic or pixellated), an optional light guide, and an MRI-compatible photo-detector, such as photo-diodes (PD), avalanche photo-diodes (APD), solid-state silicon photomultiplier (SiPM or SSPM), or a multi-channel plate, or similar detectors immune to magnetic fields.

Referring to FIG. 2, a semiconductor imaging unit, CDU 24, according to an embodiment of the present invention invention, includes a semiconductor substrate (or crystal) 20 (see, e.g., FIG. 1) for producing charge carriers (electrons and holes) through interaction with gamma photons. The CDU 24 is intended to be small in volume relative to the open space in the RF coil and the volume of space surrounding an object 14 (e.g., a patient); this small volume allows multiple CDUs 24 to be employed, each capable of providing a unique angular perspective (projection view) of the object 14 being imaged, thereby acquiring a set of projections for tomographic reconstruction.

The principle of operation of a semiconductor detector is the following: if a gamma photon (e.g., from the patient or the object being image) penetrates the detector it produces electron-hole pairs along its track, the number being proportional to the energy loss. An externally applied electric field separates the pairs before they recombine; electrons drift toward the anode, holes to the cathode; the charge is collected by the electrodes (charge collection). The collected charge produces a current pulse on the electrode, whose integral equals the total charge generated by the incident particle, i.e., is a measure of the deposited energy. The readout goes through a charge-sensitive preamplifier, typically followed by a shaping amplifier.

One embodiment of the present invention includes pixellated semiconductor imaging modules made of CZT. However, the semiconductor imaging module does not necessarily have to be CZT, and it can be another compound semiconductor such as silicon (Si), germanium (Ge), cadmium telluride (CdTe), mercuric iodide (HgI₂), thallium bromide (TlBr), gallium arsenide (GaAs), or cadmium manganese telluride (CdMnTe). In one embodiment, these modules are square and planar and can be tiled to form a line or a rectangular mosaic of modules. In an aspect of an embodiment of the present invention, the semiconductor is configured to not substantially interrupt the operation of the MRI components, and/or the strong magnetic field is configured to not substantially disturb the functionality of the semiconductor detector. Having both modalities capable of simultaneous or adjacent and sequential imaging can thus be realized.

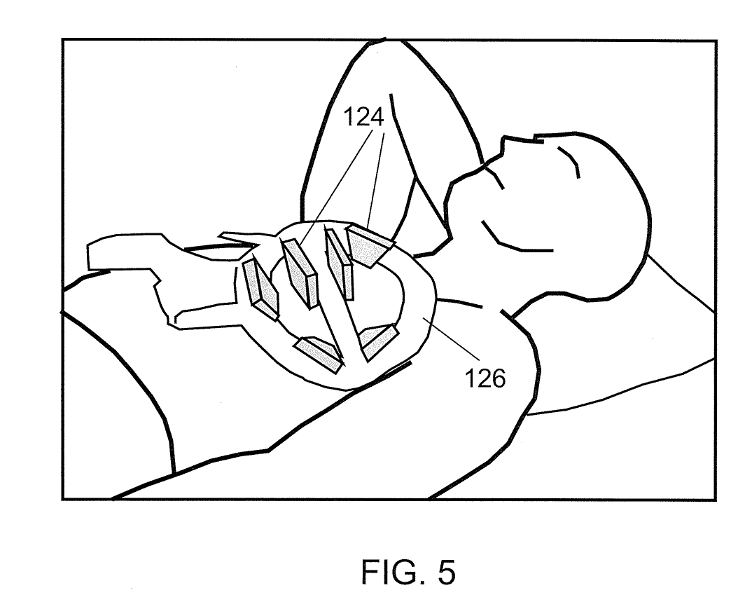

In order to perform tomographic imaging, the CDUs 24 have to sufficiently sample various angular directions. FIG. 4 shows a depiction of four individual CDUs 24 arranged at different angular orientations relative to each other, thereby demonstrating the ability to acquire tomographic data while imbedded within an RF coil 26.

In more detail, FIG. 1 depicts the general composition of a SPECT imaging unit, CDU 24. The CDU 24 is composed of a semiconductor detector 20 formed of an array of pixellated CZT modules. In FIG. 1, a 3×3 array of nine (9) CZT modules is shown to be confined to a planar surface. Each of these 9 modules is pixellated with an array of pixels numbering typically between 16 (4×4 pixels) and 1024 (32×32). Each CZT module is typically square with each side extending between 25 and 40 mm. The number of pixels per module and the number of modules per CDU are not fixed, and these specific numbers are given for illustration purposes only. The CZT modules of the semiconductor detector 20 of FIG. 1 do not necessarily have to lie on a plane; they can also be arranged such that the surfaces facing the object being imaged lie on a curved surface. As shown in FIG. 1, the CDU 24 includes the semiconductor detector 20 for detecting gamma photons and a collimator 17 (described in more detail below) for SPE imaging an object (or subject) under study (e.g., a human or animal body) with the semiconductor detector 20.

It will be understood by those of ordinary skill in the art that the semiconductor detectors include, in addition to the semiconductor substrate and metallic electrodes, readout electronics, which may be located adjacent to the substrate and may be packaged as a single modular unit. The readout electronics generally includes some combination of charge-sensitive preamplifiers, shaping filters, sample-and-hold circuits, analog-to-digital converters (ADC), and communication circuits. Typically, these electronics are manifested in an application specific integrated circuit (ASIC) which is connected to the various electrodes either directly (e.g., wire or solder bonds) or through an interface connection board. In some embodiments, the ADCs are part of the ASICs, whereas in other embodiments, the ADCs may be located on a separate readout circuit board some distance away from the plurality of detector modules.

FIG. 2 shows that the collimator 17 (see also FIG. 1) is located between the semiconductor detector 20 and the object 14 being imaged. The collimator 17 are typically composed of any of the suitable heavy metal elements, such as tungsten, tantalum, uranium, gold, platinum, iridium, and/or lead. Other suitable dense materials are not precluded from being used for image formation as a collimator. The gamma photons emitted from the object under study encounter the collimator 17 and cast a shadow through holes (apertures) in the collimator 17. These holes can be elongated as tubes or knife-edged (hour-glass shaped). The elongated holes form a family of collimating units known generally as “parallel-hole collimators”. The family of parallel-hole collimators is composed of several embodiments of the same basic design—perpendicular parallel hole collimators have every elongated hole parallel to each other and perpendicular to an entrance surface 19 of the collimator 17; fan-beam converging collimators point to a common line located on the object side of the collimator's gamma entrance surface 19 (and hence are not strictly parallel-hole); cone-beam converging collimators have holes that point to a common point located on the object side of the collimator's gamma entrance surface 19 (and again are not strictly parallel-hole). Diverging fan beam collimators and cone-beam collimators have the same description as their converging counterparts except their lines and points of convergence, respectively, are on a detector surface or side 21 of the collimator 17.

Pinhole collimators can be typically knife-edge or keel-edge, and can have single or multiple pinholes. The pinholes have an acceptance angle formed into the heavy metal, defining the pinhole's field-of-view within the object being imaged and the projection area on the detector. When multiple pinholes are in use, the projections of the images onto the detector can overlap or remain separate, depending upon the acceptance angles of the pinholes and other design parameters of the relative geometry of the pinhole locations relative to each other and the collimator-detector distance. Coded apertures are a kind of multiple pinhole embodiment in which many pinholes are used in a pseudo-random pattern and overlap of projection data is predominant in the operation and reconstruction of coded aperture imaging data.

The distance 11 between the collimator exit surface 21 and a detector entrance surface 23 is reduced or minimized when parallel-hole collimators are in use. The distance 11 can be several centimeters when pinholes are in use, allowing for magnification and a design trade-off between field-of-view and spatial resolution—that is, high spatial resolution can be achieved with greater magnification but at the cost of smaller field of view.

The operation of the SPECT imaging system comprised of the CDUs together with the MRI system can be simultaneous or sequential, with simultaneous acquisition having certain advantages, as discussed in Wagenaar et al. “Rationale for the Combination of Nuclear Medicine with Magnetic Resonance for Pre-clinical Imaging,” Technology in Cancer Research and Treatment, Vol. 5, pp. 343-350, which is incorporated by reference herein in its entirety.

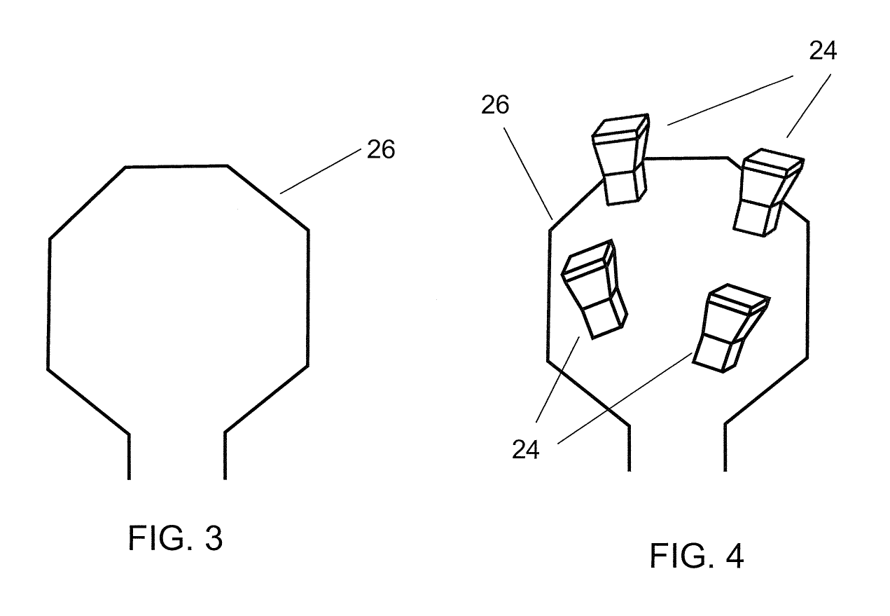

FIG. 3 depicts the fundamental unit of the RF coil (and/or transceiver) 26 found in MRI systems—a loop of conductive wire. This loop has the general shape of a perimeter with an opening located centrally, with the electrode connections coming together in close proximity to “close the loop”. The perimeter can be circular, ovoid, rectangular, or polygonal. Some loops are twisted to form a FIG. 8 shape. In typical clinical RF coils that are designed for dedicated organ-specific use (for example, brain or breast imaging), more than one loop is used to cover the surface of the body or body part (i.e., to cover the object being imaged). For example, a typical volume coil used routinely for head imaging is the so-called birdcage coil, composed of two end rings with typically 8 to 16 rungs connecting them. Geometrically, the birdcage coil can be described as composed of typically 8 to 16 overlapping loops. Multi-channel array coils, including parallel transmit and/or receive coils, are essentially composed of overlapping RF loops. Whether one or several loops are used in the RF coil design, there remains open volume between the conductive wires that can accommodate the small CDUs of FIG. 2. In RF coils that include an RF shield, the CDUs may penetrate the shield or be contained entirely within the shield.

FIG. 4 shows how four individual CDUs, each based on semiconductor detectors 20 capable of collecting projection image data through respective collimators 17, can be configured at different angular orientations such that images from four different project angles are acquired. In this way the complete tomographic dataset can be acquired. Also, the RF coil now includes the CDUs imbedded within the coil such that the RF coil with CDUs is a single accessory to the MRI system that is capable of providing SPECT and MRI imaging of the organ for which it was designed to image. The embedding of CDUs within an organ-specific RF coil such that tomographic SPECT projection data can be acquired sequentially or simultaneously with MRI data is an important aspect of an embodiment of the present invention.

FIG. 5 illustrates embedding of individual CDUs 124 with one or more cardiac RF coil 126 according to an embodiment of the present invention. Here, the individual CDUs 124 are shown to be incorporated into the cardiac RF coil 126 of the MRI system.

It will be apparent to those skilled in the art that the interactions between the MRI and SPE systems must be reduced or minimized and that any residual interactions should be compensated to render the dual-modality images relatively free from distortion, noise, and artifacts. In particular, the MRI system may need compensation for the presence of the SPE system. Electromagnetic shielding of the electronic boards of the SPE system may prevent leakage of RF noise into the RF coil of the MRI system. The presence of some conducting components in the SPE system may perturb the tuning or impedance matching of the RF coil, requiring tuning and matching adjustments for the resonant elements of the coil. The static magnetic field may be perturbed by the SPE system, requiring additional static and/or dynamic shimming of the field to increase or optimize MRI signal. The conductive components of the SPE system may sustain eddy currents in response to the magnetic field gradients used for producing MR images, requiring additional eddy-current compensation. The presence of the SPE system may increase the electromagnetic load placed on RF and gradient amplifiers, requiring compensation. The SPE system may produce heat that should be compensated by additional cooling. Any conducting power or data cables used by the SPE system may need to be filtered to prevent transmission of RF noise around the carrier frequency from entering the MRI system and degrading the image quality. Some of these effects can be reduced by using sound design engineering practices, well-known to those skilled in the art. There will likely be residual effects that should be compensated to increase or optimize the MR image quality, as suggested by the partial list of effects discussed above.

Similarly, the SPE system may need compensation for the presence of the MRI system. Because the RF coil transmits strong electromagnetic radiation, the SPE system needs shielding to operate without interference. The static magnetic field induces a Lorentz force (related to the Hall effect) on the charge carriers in a semiconductor detector operated in an MRI magnetic field. At 3 T the effect is to shift the electron cloud on average about 1.5 mm when the detector is placed with the magnetic field perpendicular to the bias electric field, which is the orientation where the Lorentz force is maximal. The presence of the MRI system may affect the electromagnetic load on the electronic components, for example during the pulsing of magnetic field gradients, requiring shielding or other compensation to reduce or minimize such interactions. The MRI system may contribute heat to the SPE system, for example during fast gradient-echo sequences such as diffusion tensor imaging, requiring additional cooling of the SPE detectors and electronics which may be sensitive to temperature fluctuations. Filtering of data and power transmission lines used by the SPE system may affect the timing and amplitude of signals, requiring compensation. Some of these effects can be reduced by using sound design engineering practices, well-known to those skilled in the art. There will likely be residual effects that should be compensated to increase or optimize the SPE image quality, as suggested by the partial list of effects discussed above.

In one embodiment of the present technique, the Lorentz force effect may be largely avoided by moving the integrated RF coil and SPE system into the fringe field of the magnet during SPE imaging and into the magnet isocenter during MR imaging. This solution will provide for interleaved (sequential) dual-modality imaging with no relative motion of the subject between scans by the two modalities. This may be particularly useful for small-animal imaging at very high field strengths, such as 9 T, where the Lorentz force effect would result in an almost 5 mm shift of the electron charge cloud in a typical 5 mm thick CZT detector module.

One advantage of combining MRI and SPECT modalities in a simultaneous imaging arrangement, as provided by the present technique, is that the information contained in the anatomically exquisite MR image can be used by those skilled in the art to improve substantially the SPECT image. In particular, the MR image can be used to derive an attenuation map appropriate for the energy of the gamma photons emitted by the radiopharmaceutical agent injected into the patient's blood stream. This ability has been developed and demonstrated by the researchers involved in pioneering the dual-modality application of PET/MRI. It is, in fact, easier and more reliable to predict attenuation of SPECT gamma photons (typically 140-365 keV) in comparison with PET gamma photons (511 keV). The attenuation map can be used to compensate the SPECT image data for both attenuation and scattering effects, resulting in a quantifiable image. In addition, the use of the MR images as prior information for the SPECT statistical reconstruction can lead to much higher resolution images with fewer reconstruction artifacts, such as aliasing.

The design of an MRI-compatible collimator is not easy, as those skilled in the art know well. However, in general, the collimator should be made of materials with good or optimized electromagnetic properties, such as conductivity and susceptibility, that do not distort the main and RF magnetic fields beyond the capability of the MRI system to compensate. For example, if the collimator is integrated into the RF coil, it must be generally transparent to RF signals, requiring minimal conductivity. If the collimator is placed close to the object being imaged, it must not distort the main magnetic field appreciably, requiring low susceptibility. Of course, the ideal design parameters can not be achieved with real materials, so optimization of the design is required.

There are many configurations conceivable for mechanically integrating MRI-compatible SPE detectors (CDUs) into RF coils, just as there are many geometries of RF coils in use and more possible for future applications. In general, embodiments of the present invention is envisioned to have CDUs that are embedded at least partially into the contiguous volume that encloses the RF coil, for example, as shown for a figure-8 cardiac surface coil in FIG. 5. Conversely, there are envisioned designs in which, for example, CDUs are formed into a contiguous ring and the RF coil elements of a multi-channel array coil (or parallel transmit/receive coil) are mechanically integrated into the contiguous volume enclosing the ring of CDUs. The mechanical integration may also include a coupling or support, either rigid or allowing constrained motion, in which either the SPE system elements (CDUs) are supported by the RF coil structure or vice versa.

While the invention has been described in connection with certain exemplary embodiments, it is to be understood by those skilled in the art that the invention is not limited to the disclosed embodiments, but, on the contrary, is intended to cover various modifications included within the spirit and scope of the appended claims and equivalents thereof.

What we claim is:

1. A combined magnetic resonance imaging (MRI) and single-photon emission (SPE) imaging system, the system comprising:
an MRI system comprising at least one SPE-compatible radiofrequency (RF) coil, the MRI system being for magnetic resonance (MR) imaging of an object; and
an SPE imaging system comprising at least one MRI-compatible gamma photon detector and at least one MRI-compatible collimator, the SPE imaging system being for SPE imaging of the object;
wherein the at least one SPE-compatible RF coil is mechanically integrated with the at least one MRI-compatible gamma photon detector and/or the at least one MRI-compatible collimator.

2. The system of claim 1, wherein the MRI system and the SPE imaging system of the combined MRI and SPE imaging system are configured to produce sequential and/or simultaneous images of the object.

3. The system of claim 1, wherein the SPE imaging system is configured to operate inside an imaging magnetic field of the MRI system.

4. The system of claim 1, wherein the SPE imaging system is configured to operate outside the MRI system and positioned in a fringe magnetic field of the MRI system.

5. The system of claim 1, wherein the SPE imaging system is configured to produce at least one projection image and/or at least one SPE computed tomographic (SPECT) image.

6. The system of claim 1, wherein the MRI system comprises a compensator configured to compensate for the presence of the SPE imaging system, the compensator comprising an electromagnetic shield, a resonant element tuner, a static and/or dynamic magnetic field shimmer, an eddy-current compensator, an electromagnetic load compensator, a cooler, a power transmission filter, and/or a data transmission filter.

7. The system of claim 1, wherein the at least one SPE-compatible RF coil is selected from the group consisting of surface coil, volume coil, multi-channel array coil, parallel transmit coil, and parallel receive coil.

8. The system of claim 1, wherein the SPE imaging system comprises a compensator configured to compensate for the presence of the MRI system, the compensator comprising an electromagnetic shield, a Lorentz effect compensator, an electromagnetic load compensator, a cooler, a power transmission filter, and/or a data transmission filter.

9. The system of claim 1, wherein the at least one MRI-compatible gamma photon detector comprises a direct-conversion substrate material selected from the group consisting of silicon (Si), germanium (Ge), cadmium telluride (CdTe), mercuric iodide (HgI₂), thallium bromide (TlBr), gallium arsenide (GaAs), cadmium zinc telluride (CdZnTe or CZT), and cadmium manganese telluride (CdMnTe).

10. The system of claim 9, wherein the at least one MRI-compatible gamma photon detector comprises:
at least one direct-conversion substrate for producing charge carriers through interaction with gamma photons; and
a plurality of electrodes for collecting the charge carriers.

11. The system of claim 1, wherein the at least one MRI-compatible gamma photon detector comprises:
at least one scintillator substrate for producing optical photons through interaction with gamma photons; and
at least one MRI-compatible optical photon detector for producing an electrical signal.

12. The system of claim 11, wherein the at least one MRI-compatible optical photon detector comprises photodiodes, solid-state photomultipliers, and/or multi-channel plates.

13. The system of claim 1, wherein the MRI system is configured to provide information to the SPE system to improve a SPE computed tomographic (SPECT) image reconstruction, the SPE system comprising an attenuation compensator, a scattering compensator, and/or a statistical reconstructor.

14. The system of claim 1, wherein the at least one MRI-compatible collimator is configured to have a single pinhole, multiple pinholes, parallel multiple holes, converging multiple holes, or diverging multiple holes; or is configured to be an inverse collimator composed of parallel, converging, or diverging multiple pins; or is configured to have multiple hole coded apertures, slits and/or slats; or is configured to have rotating slits and/or slats; or is configured to be an electronic (Compton camera) collimator.

15. The system of claim 1, wherein the at least one MRI-compatible collimator comprises a substrate of gamma photon attenuating material with electromagnetic conductivity and susceptibility properties that do not distort main and RF magnetic fields beyond the capability of the MRI system to compensate.

16. The system of claim 1, wherein the at least one MRI-compatible gamma photon detector and/or the at least one MRI-compatible collimator are at least partially embedded into the contiguous volume enclosing the at least one SPE-compatible RF coil.

17. The system of claim 1, wherein the at least one SPE-compatible RF coil is at least partially embedded into the contiguous volume enclosing the at least one MRI-compatible gamma photon detector and/or the at least one MRI-compatible collimator.

18. The system of claim 1, wherein the at least one SPE-compatible RF coil is supported on the at least one MRI-compatible gamma photon detector and/or the at least one MRI-compatible collimator.

19. The system of claim 1, wherein the at least one MRI-compatible gamma photon detector and/or the at least one MRI-compatible collimator are supported on the at least one SPE-compatible RF coil.

20. The system of claim 1, wherein the SPE imaging system is configured to be stationary during imaging.

21. The system of claim 1, wherein the SPE imaging system is configured to provide motion to the at least one MRI-compatible gamma photon detector and/or the at least one MRI-compatible collimator and/or the at least one SPE-compatible RF coil.

22. A method of combining magnetic resonance imaging (MRI) and single-photon emission (SPE) imaging, the method comprising:
introducing a radioactive isotope into an object;
acquiring at least one MR image or spectrum of an object utilizing an MRI system comprising at least one SPE-compatible radiofrequency (RF) coil; and
acquiring at least one SPE image of the object utilizing an SPE imaging system comprising at least one MRI-compatible gamma photon detector and at least one MRI-compatible collimator;
wherein the at least one SPE-compatible RF coil is mechanically integrated with the at least one MRI-compatible gamma photon detector and/or the at least one MRI-compatible collimator.

23. The method of claim 22, wherein the MRI system and the SPE imaging system of the combined MRI and SPE imaging system produce sequential and/or simultaneous images of the object.

24. The method of claim 22, wherein the SPE imaging system is stationary during imaging.

25. The method of claim 22, wherein the SPE imaging system provides for motion of the at least one MRI-compatible gamma photon detector and/or the at least one MRI-compatible collimator and/or the at least one SPE-compatible RF coil.

26. A device for combined magnetic resonance imaging (MRI) and single-photon emission (SPE) imaging, the device comprising at least one SPE-compatible radiofrequency (RF) coil mechanically integrated with at least one MRI-compatible gamma photon detector and/or at least one MRI-compatible collimator.

27. The device of claim 26, wherein the at least one SPE-compatible RF coil is selected from the group consisting of surface coil, volume coil, multi-channel array coil, parallel transmit coil, and parallel receive coil.

28. The device of claim 26, wherein the at least one MRI-compatible gamma photon detector comprises a direct-conversion substrate material selected from the group consisting of silicon (Si), germanium (Ge), cadmium telluride (CdTe), mercuric iodide (HgI₂), thallium bromide (TlBr), gallium arsenide (GaAs), cadmium zinc telluride (CdZnTe or CZT), and cadmium manganese telluride (CdMnTe).

29. The device of claim 26, wherein the at least one MRI-compatible gamma photon detector comprises:
at least one direct-conversion substrate for producing charge carriers through interaction with gamma photons; and
a plurality of electrodes for collecting the charge carriers.

30. The device of claim 26, wherein the at least one MRI-compatible gamma photon detector comprises:
at least one scintillator substrate for producing optical photons through interaction with gamma photons; and
at least one MRI-compatible optical photon detector for producing an electrical signal.

31. The device of claim 30, wherein the at least one MRI-compatible optical photon detector comprises photodiodes, solid-state photomultipliers, and/or multi-channel plates.

32. The device of claim 26, wherein the at least one MRI-compatible collimator is configured to have a single pinhole, multiple pinholes, parallel multiple holes, converging multiple holes, or diverging multiple holes; or is configured to be an inverse collimator composed of parallel, converging, or diverging multiple pins; or is configured to have multiple hole coded apertures, slits and/or slats; or is configured to have rotating slits and/or slats; or is configured to be an electronic (Compton camera) collimator.

33. The device of claim 26, wherein the at least one MRI-compatible collimator comprises a substrate of gamma photon attenuating material with electromagnetic conductivity and susceptibility properties that do not distort main and RF magnetic fields beyond the capability of the MRI system to compensate.

34. The device of claim 26, wherein the at least one MRI-compatible gamma photon detector and/or the at least one MRI-compatible collimator are at least partially embedded into the contiguous volume enclosing the at least one SPE-compatible RF coil.

35. The device of claim 26, wherein the at least one SPE-compatible RF coil is at least partially embedded into the contiguous volume enclosing the at least one MRI-compatible gamma photon detector and/or the at least one MRI-compatible collimator.

36. The device of claim 26, wherein the at least one SPE-compatible RF coil is supported on the at least one MRI-compatible gamma photon detector and/or the at least one MRI-compatible collimator.

37. The device of claim 26, wherein the at least one MRI-compatible gamma photon detector and/or the at least one MRI-compatible collimator are supported on the at least one SPE-compatible RF coil.
